(19) United States
(12) Patent Application Publication
(10) Pub. No.: US 2020/0020421 A1
(43) Pub. Date: Jan. 16, 2020

(54) A METHOD AND APPARATUS FOR COLLABORATIVE VARIANT SELECTION AND THERAPY MATCHING REPORTING

(71) Applicant: KONINKLIJKE PHILIPS N.V., EINDHOVEN (NL)

(72) Inventors: Nevenka Dimitrova, Pelham Manor, NY (US); Ronen Solomon, Eindhoven (NL); Keswarpu Payal, Elmsford, NY (US); Mine Danisman-Tasar, Eindhoven (NL); Moran Bentzur, Petah Tiqwa (IL); Nadav Sharabi, Petah Tiqwa (IL); Sergey Yussim, Petah Tiqwa (IL); Alexander Ryan Mankovich, Boston, MA (US); Vartika Agrawal, Cambridge, MA (US); Julie Gu, Eindhoven (NL); Iliya Fridman, Petah Tiqwa (IL); Kostyantyn Volyanskyy, Larchmont, NY (US)

(21) Appl. No.: 16/336,246

(22) PCT Filed: Sep. 29, 2017
(86) PCT No.: PCT/EP2017/074886

§ 371 Date: Mar. 25, 2019

Related U.S. Application Data
(60) Provisional application No. 62/401,319, filed on Sep. 29, 2016.

(51) Int. Cl. G16B 50/50; G16B 30/00; G16B 45/00; G16H 10/20

(52) U.S. Cl. CPC G16B 50/50; G16H 10/20; G16B 45/00; G16B 30/00

(57) ABSTRACT

A clinical genomic data processing device includes at least one microprocessor (10) and a non-transitory storage medium (12) storing instructions to implement functions of the device. A user interface (26, 28) receives requests for execution of genomic workflows and to display output generated by the execution of the genomic workflows. A genomic workflow manager manages an asynchronous messaging queue (24) and manages the execution of the genomic workflows. Service providers (20) performs jobs associated with the genomic workflows. The genomic workflow manager communicates with the service providers by messages exchanged via the asynchronous messaging queue to manage the execution of the genomic workflows via jobs performed by the service providers. The service providers may include a genomic processing service provider (201), an annotation service provider (202), an aberration prioritization service provider (203), a reporting service provider (204), a clinical trial matching service provider (205), and so forth.

This application claims the benefit of U.S. Provisional Application No. 62/401,319 filed Sep. 29, 2016. U.S. Provisional Application No. 62/401,319 filed Sep. 29, 2016 is incorporated by reference herein in its entirety.

### FIELD

The following relates generally to the clinical testing arts, genomic testing arts, genomic data processing architecture arts, and related arts.

### BACKGROUND

Genomics is a powerful tool for medical diagnosis, treatment selection, and other clinical tasks. In the last 15 years, since the first published map of the Human genome, the introduction of next generation sequencing has enabled interrogation of structural and functional variations across the entire human genome. The rate at which the cost of sequencing has fallen as a function of time has far surpassed the rate of integrated circuit miniaturization predicted by Moore's law. Recent large efforts such as the 1000 Genomes which mapped human genome variation across different populations, and The Cancer Genome Atlas which mapped tumor biology across 40 tissue types have stimulated biomedical research with great potential impact on the diagnosis and treatment of cancer and other ailments. Yet challenges remain in bringing genomic sequencing into common usage in clinical practice, and in effectively leveraging genomic sequencing data to yield actionable clinical information.

The following discloses a new and improved systems and methods.

### SUMMARY

In one disclosed aspect, a clinical genomic data processing device comprises at least one microprocessor and a non-transitory storage medium storing instructions. These include: instructions readable and executable by the at least one microprocessor to implement a user interface configured to receive requests for execution of genomic workflows and to display output generated by the execution of the genomic workflows; instructions readable and executable by the at least one microprocessor to implement a genomic workflow manager configured to manage an asynchronous messaging queue and to manage the execution of the genomic workflows; and instructions readable and executable by the at least one microprocessor to implement service providers configured to perform jobs associated with the genomic workflows. The genomic workflow manager is configured to communicate with the service providers by messages exchanged via the asynchronous messaging queue to manage the execution of the genomic workflows via jobs performed by the service providers.

In another disclosed aspect, a non-transitory storage medium stores instructions readable and executable by at least one microprocessor to perform clinical genomic data processing. The instructions include: instructions readable and executable by the at least one microprocessor to implement a user interface configured to receive requests for execution of genomic workflows and to display output generated by the execution of the genomic workflows; instructions readable and executable by the at least one microprocessor to implement a genomic workflow manager configured to manage an asynchronous messaging queue and to manage the execution of the genomic workflows; and instructions readable and executable by the at least one microprocessor to implement service providers configured to perform jobs associated with the genomic workflows. The service providers include at least one genomic processing service provider configured to perform a job comprising processing genomic data to generate a list of aberrations, at least one annotation service provider configured to perform a job comprising processing a list of aberrations to generate annotated aberrations, at least one aberration prioritization service provider configured to perform a job comprising processing a list of annotated aberrations to generate a prioritized list of annotated aberrations, and at least one reporting service provider configured to perform a reporting job comprising at least display of a list of annotated aberrations via the user interface and receipt of a clinical report via the user interface. The genomic workflow manager is configured to communicate with the service providers by messages exchanged via the asynchronous messaging queue to manage the execution of the genomic workflows via jobs performed by the service providers.

In another disclosed aspect, a clinical genomic data processing method is disclosed. Via a web-based user interface, requests are received for execution of genomic workflows and output generated by the execution of the genomic workflows is displayed. Via service providers implemented on a cloud-based platform comprising microprocessors, jobs associated with the genomic workflows are asynchronously performed. Via a genomic workflow manager implemented on the cloud-based platform, state machines representing the genomic workflows are maintained, and communication with the service providers is performed by messages exchanged via an asynchronous messaging queue to manage the execution of the genomic workflows via the jobs asynchronously performed by the service providers. The genomic workflow manager further updates states of the state machines in accord with messages received from the service providers via the asynchronous messaging queue indicating successful completion of the jobs performed by the service providers.

One advantage resides in providing clinical genomic data processing devices and methods that are more effectively integrated with clinical workflows.

Another advantage resides in providing clinical genomic data processing devices and methods with a service-oriented architecture (SOA), preferably cloud-based, which employs service providers that can be frequently updated to implement the latest clinical knowledge (e.g. most up-to-date aberration definitions, most up-to-date annotation databases, current information on upcoming and in-progress clinical trials, latest therapy information, and so forth) without taking the clinical genomic data processing offline.

Another advantage resides in providing clinical genomic data processing devices and methods with an SOA architecture, preferably cloud-based, which employs service providers to perform jobs associated with genomic workflows and further provides a genomic workflow manager that manages an asynchronous messaging queue for communicating with the service providers to enable asynchronous parallel processing of various workflow tasks.

Another advantage resides in providing clinical genomic data processing devices and methods with an improved user interface for presenting the most clinically relevant genomic aberrations to clinicians.

Another advantage resides in providing clinical genomic data processing devices and methods with improved patient data security.

Another advantage resides in providing clinical genomic data processing devices and methods with an improved user interface that reduces the need to cut-and-paste information between processing components

Another advantage resides in providing clinical genomic data processing devices and methods providing processing of genomic data to generate clinically actionable information with improved computational efficiency.

A given embodiment may provide none, one, two, more, or all of the foregoing advantages, and/or may provide other advantages as will become apparent to one of ordinary skill in the art upon reading and understanding the present disclosure.

### BRIEF DESCRIPTION OF THE DRAWINGS

The invention may take form in various components and arrangements of components, and in various steps and arrangements of steps. The drawings are only for purposes of illustrating the preferred embodiments and are not to be construed as limiting the invention. In drawings presenting log or service call data, certain identifying information has been redacted by use of superimposed redaction boxes.

FIG. 1 diagrammatically shows an illustrative cloud-based clinical genomic data processing device.

FIGS. 2A and 2B diagrammatically illustrate an overall framework of a workflow for a pathologist supported by microservices.

FIG. 3 diagrammatically shows an illustrative embodiment of the annotation service provider.

FIG. 4 diagrammatically shows an illustrative embodiment of the aberration prioritization service provider.

FIG. 5 diagrammatically shows an illustrative embodiment of the trial matching service provider.

FIGS. 6-11 show illustrative displays suitably produced by the reporting service providers and displayed via the user interface of the clinical genomic data processing device.

FIG. 12 shows an illustrative display suitably produced by the trial matching service provider and displayed via the user interface of the clinical genomic data processing device.

FIG. 13 shows an illustrative display suitably produced by the therapy matching service provider and displayed via the user interface of the clinical genomic data processing device.

FIG. 14 diagrammatically shows an illustrative embodiment of the reporting service provider.

### DETAILED DESCRIPTION

A difficulty with leveraging genomics in clinical practice is a dearth of informatics to store, manage, analyze and contextualize this data in a streamlined way that supports the clinical workflow of the clinical experts like oncologists and pathologists. The challenge is that there are many therapeutic options and many clinical trials and it is hard to test for one gene at a time. Next Generation Sequencing (NGS) platforms provide an opportunity to sequence genomes in a high throughput manner at reasonable cost. Algorithms exist that generally convert genomic data into meaningful biological information. Such algorithms are typically geared towards the bioinformatics expert user. Clinical specialists spend decades in obtaining specific expertise and forming their approach to problem solving and helping patients. In their way of thinking, the informatics tools they use should have a natural flow keeping in mind problems to solve and pertinent information that is needed to accomplish their task. Certain tasks may involve logging into half a dozen different IT systems, manually cutting and pasting, which may reduce the visibility of the right information and increases the chances of making errors.

Accordingly, it would be desirable to provide an informatics platform that includes a user experience that presents information in a lucid, workflow supporting fashion while leveraging clinical knowledge from various resources for annotation and interpretation to address the needs of clinical experts. Various embodiments follow a philosophy that the technology should be working to reduce time, increase the productivity and the chances for great outcome for patients. Information is deeply embedded in data which is not easily accessible in the modern day EMRs, LIS, and other clinical applications. In some embodiments, seeking expert opinion from other more experienced clinicians is an available option within the context of the decision making of a single patient.

According to various embodiments, the goal is to process genomics and clinical data including imaging and pathology data as well as any other real time diagnostic inputs to provide precision diagnostics. Some clinical questions to be answered include the following: How to match a tumor's genotype with a potential therapy for best outcome? How to elucidate the cancer subtypes in a set of tumour samples characterized at genomic, transriptomic, proteomic, epigenomic and metabolomics level? How to provide a new hypothesis and diagnosis for a patient who has been through an extensive battery of tests and is still a medical mystery? How to associate the patient microbiome data with the health condition of the person?

However, converting high-throughput genomic data into clinically actionable information is not a straightforward task. A first challenge is to be able to ingest and store extremely large amounts of genomic data (up to 1 TB for a single patient whole genome) in a reliable and secure manner while satisfying legal requirements for long-term storage. A second challenge is to be able to run asynchronously parallel processing heterogeneous pipelines and associated jobs (e.g. sequence alignment, variant and mutation calling, copy number variation detection), written in various programming languages, in a highly quality controlled, reliable, reproducible and scalable manner. A third challenge is to dynamically integrate domain-specific knowledge from various databases that may require frequent updates and to generate clinically actionable results that are reproducible during subsequent runs. A fourth challenge is to enable continuous communication across clinical specialties because oncology is usually a collaborative effort. There are many different insights to be conveyed and put together both from each clinician and also the outputs of the many smart algorithms. Various embodiments disclosed herein facilitate sharing pertinent information, communicating discordance between various types of clinical evidence, and promoting problem solving both for the diagnostic process as well as for the therapeutic planning and monitoring phase of the patient care.

Various embodiments described herein utilize a software product deployed within a cloud based platform running on various hardware including processors (e.g., microprocessors, FPGAs, ASICs, etc.), memories (e.g. L1/L2/L3 cache, system memory, and storage devices), network interfaces (e.g., Ethernet, WiFi, etc.), and so forth. An aim of the software is to provide readable and interpretable genomic information, which will present suggestions for therapy planning in oncology, however could be also used for constitutive genomics and other fields.

Various embodiments disclosed herein take data output from next generation sequencing machines, and other genomics instruments along with data from various clinical information technology (IT) systems and perform functions such as the following: (1) perform many different processes at the same time for a multitude of institutions with many users with different types of roles (e.g. oncologist, geneticist, pathologist, bioinformatician, molecular specialist); (2) automated execution of specific analytic pipelines for gene panels, whole exomes and whole genomes in order to detect DNA/RNA aberrations, by utilizing bioinformatics algorithms, and integrating such information to provide the clinical experts (such as oncologists, pathologists, and medical geneticists) with user portals with guided workflows to enable the review of candidate aberrations, along with annotated information, to facilitate aberration selection (i.e., aberrations the clinician believes are associated with the disease) and clinical report generation, clinical information and/or therapeutic treatment options, based on public and/or curated private database, and descriptions and links to eligible clinical trials; (3) associations of new genomic/transcriptomic/epi-genomic/proteomic biomarkers; (4) storage of both raw and analyzed data combining results from NGS with patient demographic, diagnostic, lifestyle and outcome data; (5) storage of analytical metadata and intermediate files produced in analysis; (6) storage of end-user actions which were applied to produce a clinical report; (7) social-network like communication features between clinicians to share more case-related information and second opinions; and (8) assembling the relevant information and generating a clinical report.

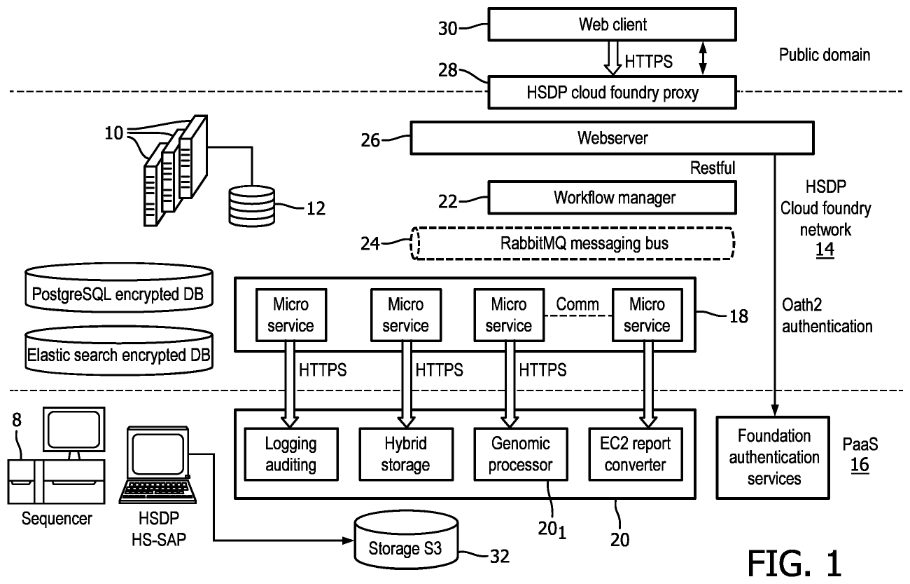

With reference now to FIG. 1, an illustrative clinical genomic data processing device is shown, which is implemented as a cloud based system. The genomic data may, for example, be acquired by a genetic sequencer 8, which preferably employ Next Generation Sequencing (NGS) to sequence genomes in a high throughput manner at reasonable cost. The cloud-based system comprises at least one microprocessor, typically implemented as one or more server computers 10 interconnected via the Internet, a wired and/or wireless local area network, or so forth, and a non-transitory storage medium 12 that stores instructions readable and executable by the least one microprocessor 10 to perform various tasks. The illustrative clinical genomic data processing device includes an architecture as shown in FIG. 1, including a Platform as a service (PaaS) 14 and a HealthSuite Digital Platform (HSDP, available from Koninklijke Philips N.V.; or other hosting platform) 16 hosting microservices 20 that the main Genomics application consumes.

An application layer sits on top of the HSDP Cloud Foundry Network 16 (or similar) and perform various functions such as: providing connections 18 to PaaS microservices 20; implementing new microservices 20 specific for oncology, for example, clinical reporting microservice, annotation microservice, therapy matching microservice, clinical trial microservice, variant prioritization microservice, variant filtering microservice, auditing and logging, identify access management, pipeline management microservice and many others; implementing a workflow manager 22 which receives requests for execution of genomic workflows, queues up jobs associated with the genomic workflows (in the illustrative embodiment, using a RabbitMQ messaging bus 24, or more generally an asynchronous messaging queue managed by the workflow manager 22) and orchestrates the execution of these jobs by the service providers 20; and provides a back end webserver 26 which executes the complicated computations in order to manage the user events and visualize complex results. In the illustrative embodiment, the webserver 26 presents a user interface 26, 28 in the form of the webserver 26 with an HSDP cloud foundry proxy 28 via which a web client 30 (such as a web browser, e.g. Google Chrome, Mozilla Firefox, Microsoft Internet Explorer or so forth, or a custom web client communicating via a secure HTTPS protocol) communicates with the illustrative clinical genomic data processing device. The web client 30 only renders output and receives requests from the user.

The instructions stored on the non-transitory storage medium 12 include: instructions readable and executable by the at least one microprocessor 10 to implement the user interface 26, 28 configured to receive requests for execution of genomic workflows and to display output generated by the execution of the genomic workflows; instructions readable and executable by the at least one microprocessor 10 to implement the genomic workflow manager 22 configured to manage the asynchronous messaging queue 24 and to manage the execution of the genomic workflows; and instructions readable and executable by the at least one microprocessor 10 to implement the service providers 20 configured to perform jobs associated with the genomic workflows. The genomic workflow manager 22 is configured to communicate with the service providers 20 by messages exchanged via the asynchronous messaging queue 24 to manage the execution of the genomic workflows via jobs performed by the service providers.

As is known in the art, the non-transitory storage medium 12 which stores instructions that are readable and executable by at least on microprocessor 10 may, by way of non-limiting illustration, comprise memories such as L1/L2/L3 cache, system memory, and storage devices such as a hard disk drive, RAID disk array or other magnetic storage medium; a solid state drive (SSD) or other electronic storage medium, an optical disk or other optical storage medium, various combinations thereof, or so forth. The cloud-based system comprises the at least one microprocessor (e.g. server computers) 10 interconnected via network interfaces (e.g., Ethernet, WiFi, etc.), and the non-transitory storage medium 12. The web client 30 is typically implemented on a desktop computer, notebook computer, mobile device such as a cellphone, tablet computer or the like, which provides a display for presenting output generated by the execution of the genomic workflows, and one or more user input devices such as a keyboard, mouse, touch-sensitive display, dictation microphone, or so forth via which a user may initiate requests for execution of genomic workflows, enter or edit clinical reports, and otherwise interact with the clinical genomic data processing device.

The illustrative service providers 20 are microservices. Microservices are considered an extension of service-oriented architectures (SOA) used to build distributed software systems. Microservices are processes that communicate with each other over a nework using lightweight protocols. A benefit of using microservices is to enhance the cohesion and decrease coupling of software. This facilitates the ability to continuously add or drop services and refactor the system. In some embodiments, all microservices are stateless and share nothing. Any data that needs to persist must be stored in a stateful backing service, typically a database such as a cloud-based storage 32, e.g. Amazon Simple Storage Service (S3, available from Amazon Web Services, Inc.) in the illustrative embodiment. Microservices may declare all dependencies, completely and exactly, via a dependency declaration manifest. Furthermore, a dependency isolation tool may be used during execution to ensure that no implicit dependencies “leak in” from the surrounding system. The full and explicit dependency specification is applied uniformly to both production and development. The clinical genomic data processing device can have a configuration server (for example Spring Batch) and a Git repository (or similar type of software repository) that will hold the configuration for all micro services. The configuration server may be provided by a cloud foundry (e.g. the illustrative HSDP cloud foundry 14) or another, proprietary instance.

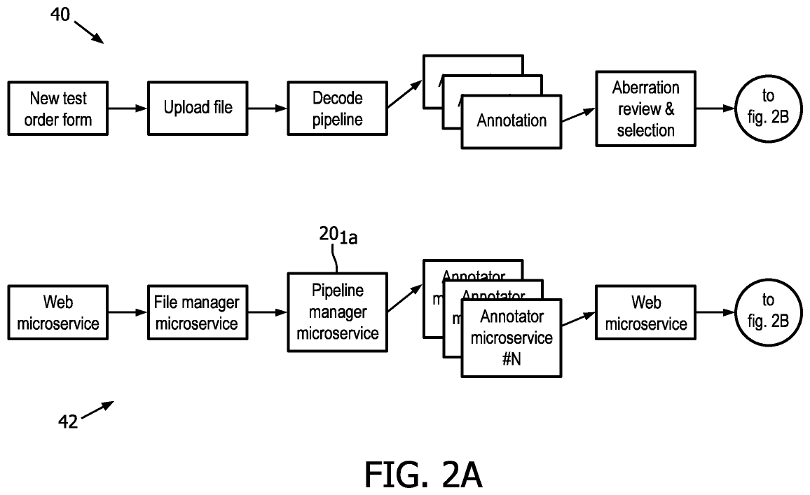

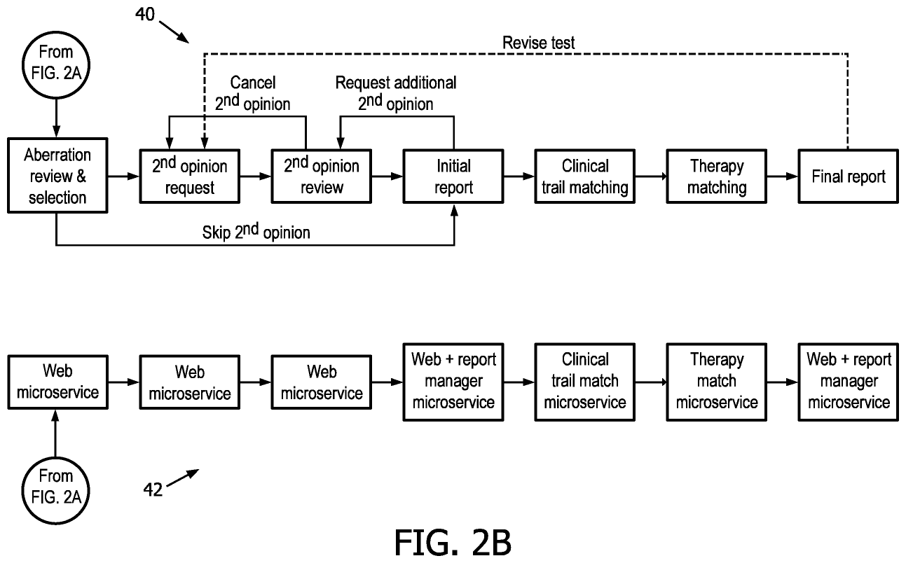

With reference to FIGS. 2A and 2B, an overall framework is illustrated of how the workflow for a pathologist (or in a similar way for an oncologist) is supported by the microservices 20. In FIGS. 2A and 2B, the top flow shows an illustrative execution of a genomic workflow 40, while the bottom flow represents a sequence of jobs 42 performed by microservices that are associated with (i.e., operate under management of the genomic workflow manager 22 to execute) the genomic workflow 40. The illustrative genomic workflow of FIGS. 2A and 2B is exemplary, one could also contemplate a different order of executing these microservices, for example, therapy match service and clinical trial service could be used in the opposite order.

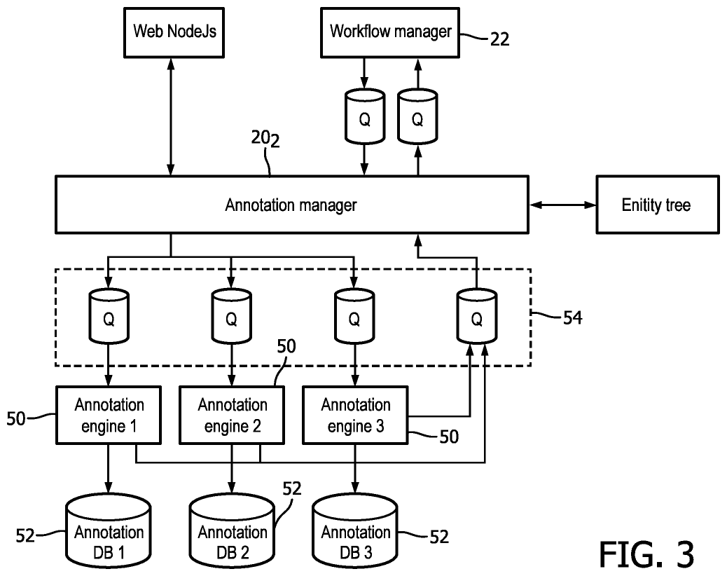

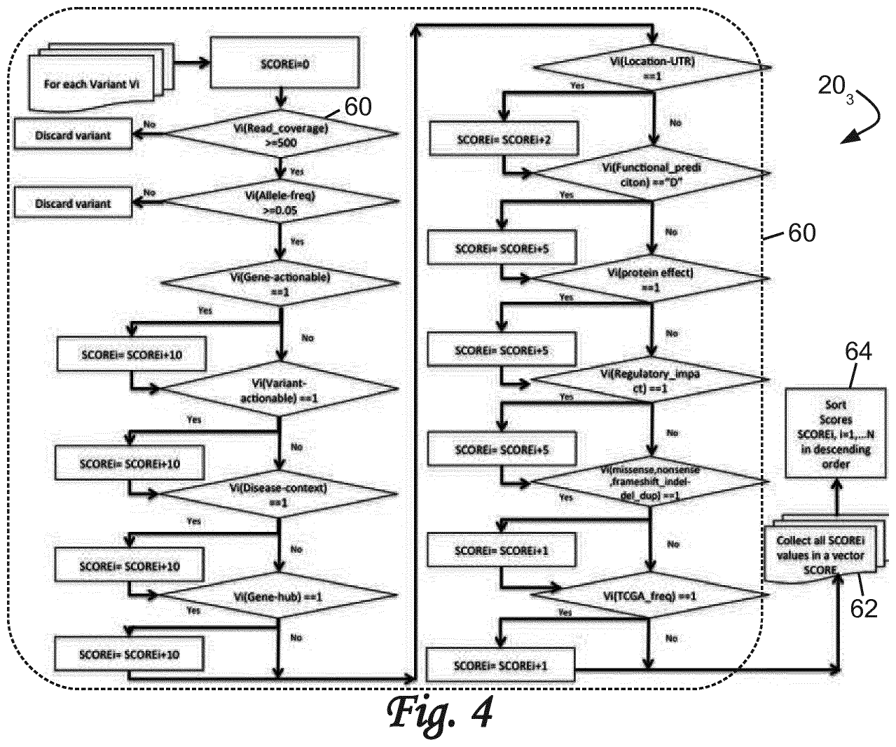

In the following, examples of various illustrative service providers 20 are described. Some of the illustrative microservices include: at least one genomic processing service provider 20₁ configured to perform a job comprising processing genomic data to generate a list of aberrations (see FIG. 1); at least one annotation service provider 20₂ configured to perform a job comprising processing a list of aberrations to generate annotated aberrations (see FIG. 3); at least one aberration prioritization service provider 20₃ configured to perform a job comprising processing a list of annotated aberrations to generate a prioritized list of annotated aberrations (see FIG. 4); at least one reporting service provider 20₄ configured to perform a reporting job comprising at least display of a list of annotated aberrations via the user interface 26, 28 and receipt of a clinical report via the user interface 26, 28 (see FIGURE); and at least one trial matching service provider 20₅ configured to perform a job comprising comparing the list of annotated aberrations to at least one clinical trial database to generate at least one clinical trial recommendation. In addition to or in place of the trial matching service provider 20₅, at least one therapy matching service provider (not shown) may be provided which is similarly configured to perform a job comprising comparing the list of annotated aberrations to at least one clinical therapy database to generate at least one clinical therapy recommendation.

With returning reference to FIG. 1, some illustrative embodiments of the genomic workflow manager 22 are described. The workflow manager 22 executes all scheduling of the different jobs that run on (i.e. are performed by) microservices 20. The illustrative workflow manager 22 exposes Representational State Transfer Application Program Interfaces (REST API's) to its clients (via the webserver 26 as shown in FIG. 1) which allows clients to request execution of genomic workflows.

The workflow manager 22 enables the workflows to be interpreted as state machines. Each step in the state machine is a job work item (e.g., a piece of software code) to be processed. The workflow manager 22 manages workflows—it does not perform any task by itself but rather relies on different job providers 20 for performing the specific jobs. When a workflow request arrives it is stored in a persistence layer and processed. The first job item is sent via the queue 24 to the specific provider 20 which supplies it. Once an item has successfully processed by a provider 20 it notifies the workflow manager 22 via the queue mechanism 24. At this point the workflow manager 22 updates the state of the state machine and sends the second job in the request to the second job provider 20 and so on until all the jobs are done or there was a failure. At that point the workflow manager 20 updates the status of the executing workflow with success or failure for the step performed by the completed job(s). The illustrative clinical genomic data processing device takes into consideration that both the workflow manager 22 and its providers 20 are microservices and that, at any point in time, a job may be handled by a different workflow manager or by provider instances. The workflow manager 22 will thus use the microservices cloud infrastructure services.

With continuing reference to FIG. 1, some suitable embodiments of the genomic processing service provider 201 is next described. The microservice 20₁ for genomics processing is triggered automatically for every test when new input data is available on a file server or on a sequencer drive of the genomic sequencer 8 to which the clinical genomic data processing device has access to check automatically for the end of a sequencing run. Each test which is ordered is associated with a well-defined clinical pipeline which has been developed as part of a genomics laboratory validation process, or as part of an in-vitro diagnostics (IVD) test. All the tools, all the parameters for the pipeline are fixed and are consistently applied across all the samples. Genomics processing may be performed using various genomics processing platforms such as, for example, the PAPAYA genomics platform which processes sequencing data, for example in a FASTQ format, by operations such as alignment and variant calling to generate a list of aberrations which may for example be stored in a variant call format (vcf format). One of the processes that deals with the pipeline during the operation of the genomic processing service provider 20₁ is a Pipeline Manager 20₁ₐ (see FIG. 2A). The pipeline manager microservice 20₁ₐ a runs pipelines and monitors their execution. The pipelines are stored and executed on specific engines such as a genomics platform (e.g. PAPAYA). The pipeline manager 20₁ₐ exposes all available pipelines and missions via a REST API. The execution request of a pipeline and the reception of its completion are performed via the asynchronous messaging queue 24 (which is a RabbitMQ message broker in the illustrative device of FIG. 1). In order to pull an ongoing execution the pipeline manager 20₁ₐ may use a delay queue that will send timed messages to the pipeline manager 20₁ₐ to check on a pipeline execution status. This is particularly advantageous in a typical clinical deployment in which thousands of such requests may be received per minute, and where each such request may be critical for patient care. The illustrative pipeline manager 20₁ₐ is implemented via the microservices cloud infrastructure.

With reference now to FIG. 3, some suitable embodiments of the annotation service provider 20₂ is next described. Genomics annotation is the next step towards interpretation of genomic data and converting genomic aberration locations into usable information for doctors and researchers. In various embodiments of the clinical genomic data processing device, the annotation manager service provider 20₂ receives a request from the workflow manager 22 to perform annotations on a set of genomic aberrations. This is triggered with the knowledge within the system that specific annotation type is run within a particular next generation sequencing test type (or another genomics test) for a clinician (oncologist or pathologist) or a biologist/molecular specialist. The annotations manager (i.e. service provider) 20₂ receives a list of aberrations (for example in a vcf format) with the identification (ID) of the specific workflow/revision and then according to the requested annotation type the annotation manager 20₂ runs annotation engines 50. These annotation engines 50 can bring in knowledge from publicly available resources 52 such as UCSC genome browser, ClinVar, ClinGen, dbNSFP, COSMIC, TRANSFAC, 1000 genomes project, TCGA database, KEGG pathway database or so forth. Annotation with each one of these resources 52 may be executed as a separate job. In addition, each type of genomic test (for example, a somatic mutation test) may have a separate combination of Genomics Annotation resources, and this is optionally configurable at the system level: to associate a type of test (e.g. TruSeq48) with a specific pipeline and a specific set of annotation resources. For example, if there is a 48 gene panel for somatic mutations (a cancer test) the type of annotations would include: UCSC, COSMIC, dbNSFP, while a germline mutation test (from normal sample) the type of annotations would include: UCSC, dbNSFP, KEGG pathway and ClinVar.

Once the annotation manager 20₂ receives an annotation match request it may perform one or more of the following steps. (1) Receive all genomic aberrations (SNV, CNV, fusions) for the requested workflow process. (2) Retrieve a list of all available annotation sources and their respective latest active versions (unless specified otherwise). (3) Create a progression entry for each annotation source in order to mark the progress of annotation with that particular source. (4) Send annotation match request to a specific service called vcfEtl, which is responsible for fetching and transformation of the entries of the vcf file into annotated entries, one per annotation source, with each row representing another genomic aberration. (5) Send an acknowledgement to the messaging broker 54 (messaging is asynchronous, decoupling applications by separating sending and receiving data). (6) After this point, the annotation match requests are processed by vcfEtl instances and upon completion they send annotation match responses with a body of the annotation results. (7) When receiving annotation match response the annotation manager 20₂ updates the progression entry for the source that responded. At this stage it checks that this response was not already received and failed due to error. However, if there was an error in the past the annotation manager 20₂ performs a database clean-up of the annotation results and another attempt to reprocess the response. (8) The annotation results for this source are stored in the database as annotated results. (9) The entry noting the progress for this source is updated to “done”. (10) The annotation manager 20₂ checks if all match sources returned successfully using the progression entries. If the match resources have not yet returned successfully, then it waits, and if some failed it returns a “fail” to the workflow manager 22. If all are successful then it returns a job done with success status to the workflow manager 22. (11) After this, the annotation results become available for the next steps of the genomic workflow, for example displaying results via the user interface 26, 28 or for submitting these results for therapy and clinical trial matching.

Once all annotation engines 50 have notified the annotation manager 20₂ they are done the annotation manager 20₂ creates the annotation entries, and sends a notification to the workflow manager 22 that the annotation job has been done and all results are available to be retrieved.

Because biological and clinical knowledge is an ever growing area, new annotation databases 52 may be brought into the engines 50 to update the annotation capabilities of the clinical genomic data processing device on a continuous basis. There are at least two ways: 1) a database for an annotation engine has a new version, or 2) a completely new database may be included with a novel data schema.

With reference now to FIG. 4, some suitable embodiments of the aberration prioritization service provider 20₃ is next described. For whole exome or whole genome sequencing (WES or WGS), a sample may have millions of genomic variants. Without annotation and subsequent prioritization of such variants, researchers and clinicians waste valuable time and resources on variants of no significance, rather than focusing on those variants which may be contributing to human disease. When the goal is to match a variant to a clinical trial, it is important to know if the variant exists in other datasets or is so rare that finding a matching trial is unlikely (as recruiting success for such a trial would be limited). By only selecting the important variants by the content of their associated existential, functional, and disease-related annotations, the complexity of clinical trial matching can be drastically reduced. Accordingly, one purpose of the clinical genomic data processing device is to be able to classify and prioritize variants so that the clinician can readily access and filter these variants in order to prioritize for inclusion in the clinical report. After variants have been annotated, there is a variant prioritization process (performed by the aberration prioritization service provider 20₃) based on the classification of the variants. The classification is based on the immediate impact of the variant on the function of the respective protein. The relevance is that these prioritized variants are the ones that are most likely to have impact on the creation of a therapy plan for the patient.

In the illustrative embodiment of FIG. 4, the variant prioritization is based on the priority of the type of annotation and works as follows. Variants get annotated with several types of information: quality information; actionability; disease context; location of the variant; and frequency information. These are addressed in turn in the following.

Quality information comes as part of the genomics processing pipelines 20₁ₐ (see FIG. 2A). For example, the information may include the quality of the “signal”, say quality of base calling, number of reads that cover the genomic aberration (e.g. total number of reads), variant allele frequency which signifies how many reads support the variant call (e.g. 10% of the reads at a given position are “C” which in the reference genome is “A” and give evidence to support a variant call as “C”). Variants which do not meet the quality criteria may be discarded from the prioritization process.

Actionability is based on availability of U.S. Food and Drug Administration (FDA) approved therapies or trial matches for a specific gene or specific variant.

Disease context is suitably defined as follows. For each type of cancer (in an illustrative oncology workflow), there is a priority list of genes which are very relevant for that type of cancer. For example: Jak2 for myelodisplastic syndromes, BRAF for melanomas, EGFR for lung and colon cancer. Additionally, this step could also rely on an internal database which is curated and where there is high interest in the in-house curated genes, these should be prioritized higher for the hospital where the test is being performed.

Location of the variant can be variously defined: genic (exonic, intronic, variants that a located on the 5′ untranslated gene region (5′ UTR) of 3′ UTR untranslated gene region) and intergenic. If a variant is exonic then should be prioritized by the order given above. Impact on the protein function can be considered for exonic variants: The impact classification includes non-synonymous (missense, nonsense), frameshift, insertion, deletion, duplication, indel, synonymous. Another factor may be Hub in a Pathway based prioritization: If a gene has many connections within a pathway, we will prioritize this gene higher than other genes.

For non-synonymous aberrations, the following may be considered. Functional prediction: which refer to prediction scores for deleteriousness of the variant: benign, deleterious, tolerated (or high, medium low impact on the gene function), as they are given by SIFT, PolyPhen, FATHM, MUTATIONTASTER, and others. “D” may be denoted as a score based on the values in these databases that signifies that a variant has deleterious effect on the function of that gene. Another factor may be protein effect: gain of function, or loss of function (predicted or proven) and no effect. In various embodiments, when there is effect, the annotation is 1, otherwise, the annotation is 0. Another factor may be impact on regulatory elements, such as: transcription factor binding sites, methylation sites, long-noncoding RNAs regions, microRNAs regions.

Frequency information may be based on the frequency of the variant in specific databases (for example, external knowledge bases like TCGA or internal knowledge bases). The frequency information can also be obtained from other external knowledge bases, or from the so-called beacons (https://beacon-network.org) which is a federated ecosystem for sharing genomic and clinical data as part of the Global Alliance for Genomics and Health consortium.

In the illustrative variant prioritization of FIG. 4, after the annotation process, for each one of these annotation databases, there are additional columns for each type of annotation with 1 if there is a match for the respective type of annotation or 0 if it is not. This process results in a creation of a matrix. For example Vi(Read coverage) means that the value for the i-th variant in the Read coverage column in the matrix is returned. Then, the prioritization and sorting scheme is applied as the one shown in FIG. 4. In processing 60, for each variant Vi, i=1 . . . N (where N is the number of variants in a patient case) a SCOREi is computed based on the type of annotation values for each annotation database. After all the scores are computed, they are collected in an operation 62 in a vector SCORE and in an operatoi 64 the variants are rank ordered based on the sorting of the scores in descending order. The highest ranking variants will have the highest scores. This type of ranking is especially relevant for large gene panels, exome sequencing and whole genome sequencing. One could contemplate similar scheme for annotating copy number variations or fusions, methylation events, and other genomic aberrations.

Various embodiments of the aberration prioritization service provider 20₃ may utilize additional or alternative information for filtering and/or ranking variants for display to the clinician. According to some embodiments, superset categories are defined and a score based on these supersets is assigned to each variant. These scores are used to filter and rank each variant. The categories may in one illustrative embodiment include the following, in order of importance: dataset detection, functional, disease, other evidence, which are described in turn in the following.

External/internal dataset detection is one of the more important aspects of variant prioritization in regards to treatment and clinical trial matching, the reason being that if a variant does not exist in other patients it may be unlikely a clinical trial will be designed specifically targeting that variant. Dataset detection is an annotation that results from querying external (such as the Beacon network) and internal (such as hospital IT systems) variant datasets and returns a value of ‘true’ if the variant supplied in the query exists elsewhere, and ‘false’ otherwise. In some embodiments, these datasets are chosen based on those that are sufficiently large enough (e.g., in the order of hundreds of thousands or millions) to be confident in the result. This category may return a value of 100 or 0 for ‘detected’ or ‘not detected’, respectively. This category is heavily weighted for clinical trial matching specifically.

The functional category may include annotations (which can originally range in the hundreds) indicating the functional significance of a variant. In various embodiments, only variants which are identified as non-synonymous are considered, and only annotations indicating deleteriousness/pathogenicity are weighed (such as SIFT, Polyphen-2, Mutation Assessor, Condel, FATHMM, CHASM, and transFIC cancer-impact tools). The value of each weighed annotation may be a value of 1 or 0 (or a scaled value between 1 and 0 for annotations with numeric values), depending on whether the conclusion is deleterious/pathogenic or not. This category returns the average of these values. These values may only be considered for annotations that exist in each variant.

The disease category recognizes that the presentation of a variant in human disease (such as cancer) is important for identifying clinical trials or therapies targeting that specific disease. Supplied with the disease indication of the patient, and the disease associated with the variant (an annotation sourced from databases such as ClinVar, or the Jackson Laboratory's Clinical Knowledgebase), variant priority can be decided with in the order as follows: those involved in the disease of the patient, those involved in other diseases, and those not known to have any involvement in human disease (e.g., values of 1, 0.5, and 0, respectively).

Other Evidence is a “catch-all” category. In cases where there is additional data for the sample from other genomic modalities (e.g. transcriptomics), it is possible to gain additional insight about a variant. Some functional prediction tools (e.g. Ensembl Variant Effect Predictor) supply all transcripts associated with a particular variant. However, not all of these transcripts are actively expressed. Cross-referencing transcriptomic data enables the system to assign higher priority to a variant if the transcript annotations matching the variant are being actively expressed.

For a functional annotation paradigm of ‘deleterious vs. non-deleterious’, a conservative expression threshold of 0 is set in some embodiments. According to various embodiments, if the potentially deleterious transcripts are not greater than this threshold, this category is assigned a value of 0. Otherwise, a value of 1 is assigned.

After quality filtering of low confidence variants, the sum is computed for all categories. Variants are sorted and ranked in descending order.

Various embodiments of the aberration prioritization service provider 20₃ may be implemented as a stand-alone piece of software which processes one or many variant call files (and can be modified to process any data structure containing variant data and aforementioned variant-specific and database-dependent annotations) in a single-processor or parallel schema. The aberration prioritization service provider 20₃ is situated on-site or in the cloud and the results represent a penultimate step in retrieving the enriched approved dataset of variants (where the final step is clinician approval). For the purposes of identifying potentially disease-causing or actionable variants, there are multiple disparate annotations by which one can prioritize.

One such situation is as follows: a biopsy is sequenced using the genomic sequencer 8 according to the approved laboratory protocol (for example, whole exome sequencing); the sequencing data is processed by the variant calling pipeline 20₁ₐ (see FIG. 2A; this is a process in which genomic variants are detected and output in a standard format such as vcf); variants are filtered for quality, depth, and other standard metrics; then, variants are given functional/clinical annotations by the at least one annotation service provider 20₂. The highest priority variants will automatically be those with matching FDA (or, in some embodiments, non-FDA) approved therapies either within or outside the patient's primary disease indication. There are relatively few of these variants, and if none appear in the sample the clinician is then faced with identifying the relative importance of the remaining bulk of variants. In this case the aberration prioritization service provider 20₃ intervenes and, according to the categorical weights provided, ranks the remaining variants by prioritizing as described. Due to the costs and complexity of variant-based clinical trial matching, the clinician may only want to select the most likely (i.e., highest ranking) matches as candidates.

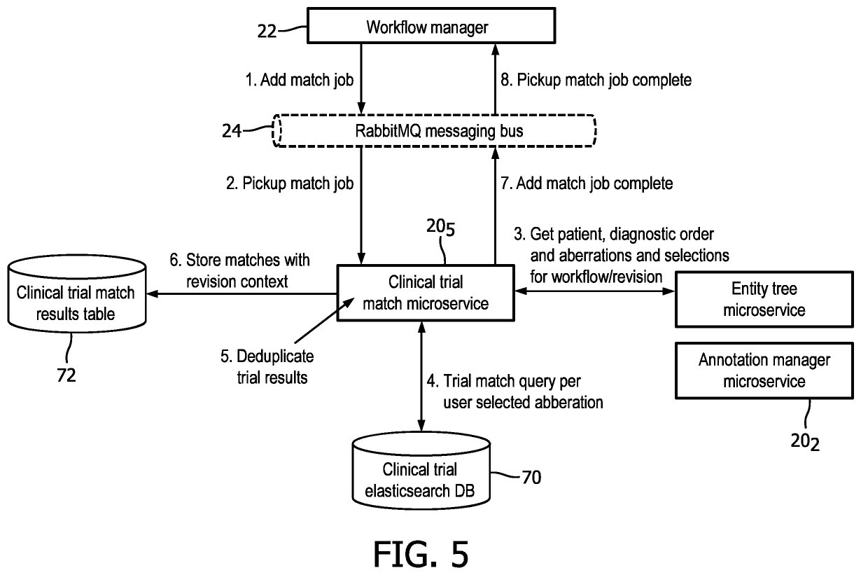

With reference now to FIG. 5, some suitable embodiments of the trial matching service provider 20₅ is next described. The clinical trial match microservice 20s provides a clinical trial matching job that can be executed as part of a genomics workflow. The clinical trial match microservice 20₅ accepts new job requests from the asynchronous messaging queue 24 and provides job completion messages on the shared workflow manager queue 24. Once a match job is initiated the trial matching service provider gathers from other services (e.g. the annotation microservice 20₂) the information needed to build a query. The service then executes a query against the clinical trial database 70 (for example a downloaded version of clinicaltrials.gov, or a private database of clinical trials that exists within a hospital or a cancer center) per selected genomic aberration (e.g. single nucleotide variant), pools and deduplicates the results. The results are saved in the entity DB 72 with a revision context. The service also provides a REST API to query for clinical trial matches based on a test revision ID.

Illustrative embodiments of the trial matching service provider 20s are described with reference to FIG. 5. It will be appreciated that a therapy matching service provider (or providers) may be similarly constructed, with the clinical trial database 70 suitably replaced by a database of clinical therapies that may be suitable for the patient.

In the following, some suitable embodiments of reporting service providers 20₄ are next described.

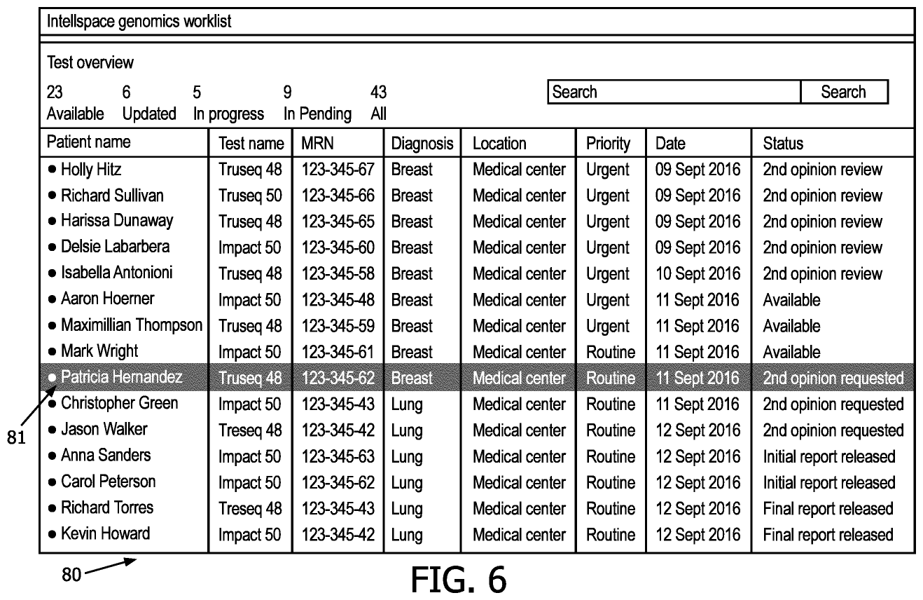

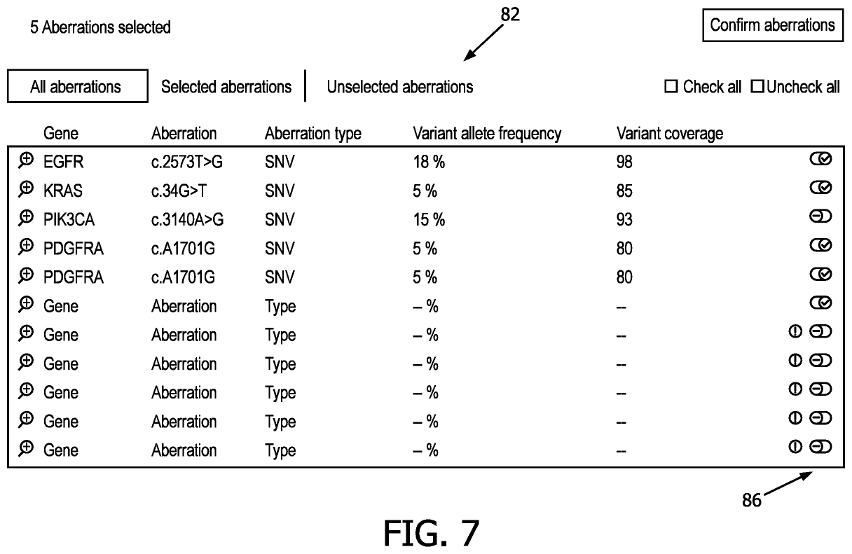

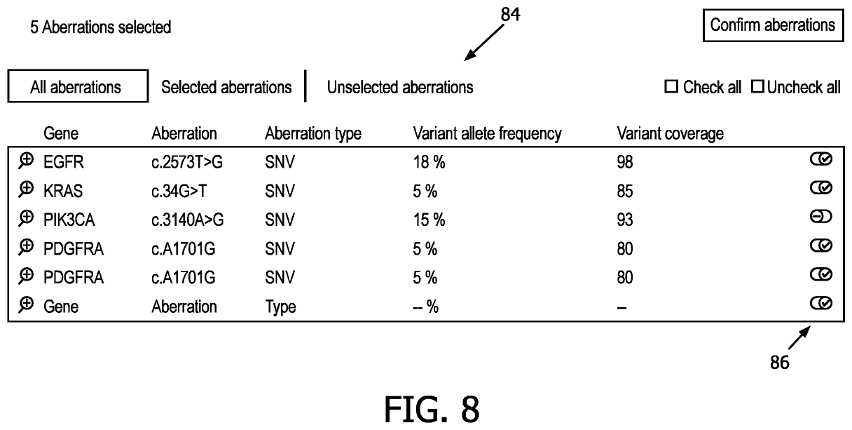

With reference to FIGS. 6-8, after login, the pathologist obtains a worklist with list of cases 80 assigned to the pathologist. The case list 80 shows the status of pending tests (whether it is still processing, sent out for second opinion, initial report and final report), high level details of cases like patient name, Medical Record Number (MRN), diagnosis, priority status and date when the test was ordered. After selecting a case, the pathologist is presented with a list of annotated variants 82 as shown in FIG. 7. For each variant a set of characteristics is shown: Gene name, the type of aberration, variant allele frequency, variant coverage. Various levels and portions of the aberrations list can be displayed. For example, upon opening a magnifying glass control (not shown) all the other information from the different annotation resources is also shown. In FIG. 8, a prioritized list 84 of only the highest-priority aberrations is shown. As can be seen in FIGS. 7 and 8, a set (illustrative column) of selectors 86 is provided via which the pathologist can select (or deselect) aberrations for inclusion (or not to be included) in the clinical report.

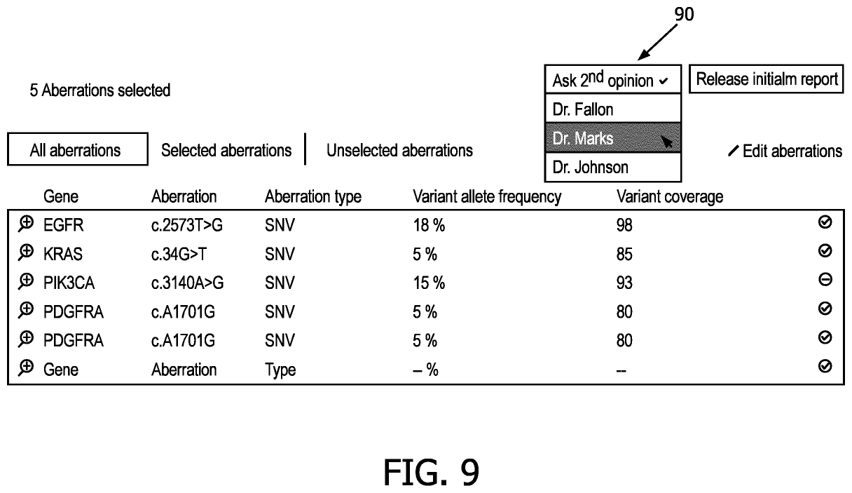

With reference to FIGS. 6-11, when there is a new or difficult case with many novel variants or one where the patient has already had multiple lines of therapy, the primary pathologist, who is reporting on the genomic test, can choose to request a second opinion from anyone who is a registered user on the clinical genomic data processing device. To do this, the non-transitory storage medium 12 (see FIG. 1) stores a list of registered users of the clinical genomic data processing device, and the reporting job performed by the one or more reporting service providers 20₄ includes display of the list 82, 84 of annotated aberrations (as per FIG. 7 and/or FIG. 8) to the first registered user (e.g. the primary pathologist) via the user interface 26, 28 (e.g. as per FIG. 7 and/or FIG. 8). A request for a second opinion is initiated by the first registered user, for example via a graphical user interface (GUI) dialog 90 (see FIG. 9) via which the first registered user (e.g. primary pathologist) can select a second registered user who will be asked to render the second opinion. This request is sent to the second registered user (who is different from the first registered user) via the user interface 26, 28. The second opinion is received from the second registered user via the user interface 26, 28 and is displayed to the first registered user via the user interface. This is an optional step in the workflow and not mandatory for all cases.

With continuing reference to FIGS. 6-11, once the (primary or first) pathologist selects the “Ask 2nd Opinion” drop box 90 (see FIG. 9), a list of qualified personnel, who are registered pathologists and oncologists appears and any one of them can be selected. Along with Genomic aberrations, notes can be typed up in a specialized window (not shown) and also sent to the second opinion provider (i.e. second registered user) in the context of the case. Upon request for the second opinion, the status of test 81 in the worklist 80 (d) changes to “2nd opinion requested” as shown for illustrative purposes in the worklist 80 of FIG. 6.

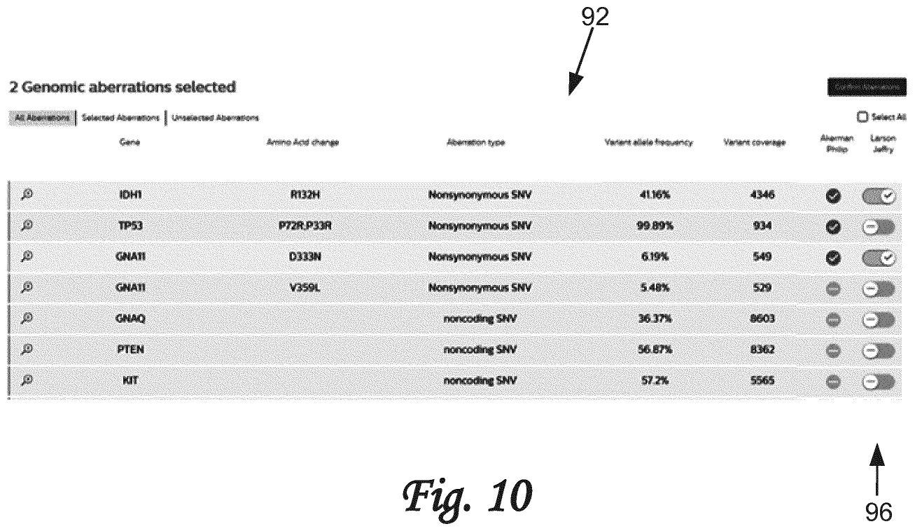

The pathologist receiving a second opinion request (i.e. the second registered user) has a similar application screen as the requesting pathologist, as shown in FIG. 10. The variant selection made by the primary reporting pathologist is optionally available for viewing (e.g., similar to the annotated aberrations list 82, 84 of FIGS. 7 and/or 8); alternatively, if it is desired for the second opinion to be “blind”, so as not to be biased by the analysis of the primary pathologist, then this information may be unavailable to the second registered user. The second opinion pathologist (i.e. second registered user) provides a selection of variants in a similar fashion as described for the first pathologist with reference to FIGS. 7 and 8. To this end, the displayed list of annotated aberrations 92 shown to the second opinion pathologist includes a set (illustrative column) of selectors 96 analogous to the set of selectors 86 provided to the primary pathologist. The second pathologist may also be provided with a messaging interface to type and send notes along with the variants selected.

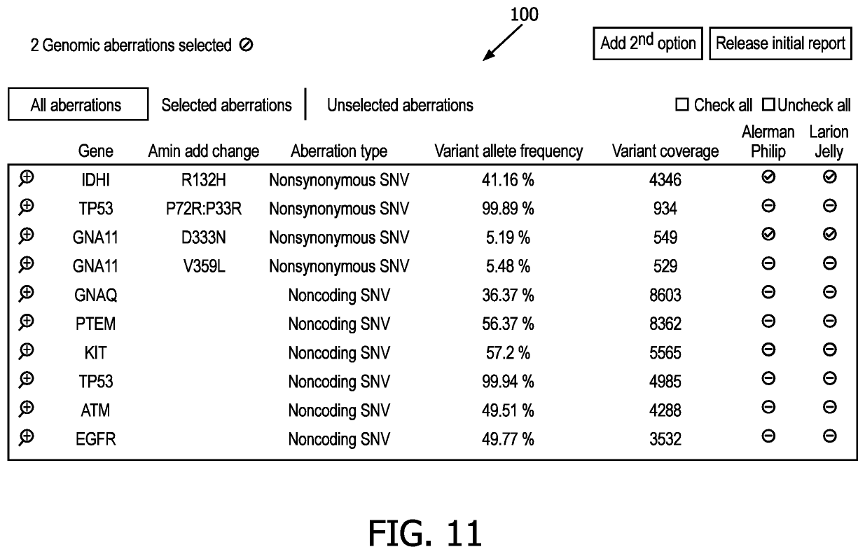

After selection of the second opinion aberrations is confirmed by the clinician, the one or more reporting service providers 20₄ automatically adjusts both worklists to the combined list of selected aberrations 100 shown in FIG. 11. This appears as second opinion received in the primary reporting pathologists' worklist, and optionally may disappear from the second opinion clinicians' worklist (as the second opinion task is now complete).

After receiving the second opinion from a second registered user, the reporting pathologist (i.e. primary pathologist, i.e. first registered user) can again access the case from his/her worklist 80 (see FIG. 6) and then modify their own findings using an edit button or other selector to initiate report editing. At this stage, both the pathologist's selections are displayed in the application window (combined selected aberrations list 100 as shown in FIG. 11). In the same manner, the system can help the primary pathologist to add additional second opinions if desired by the primary pathologist (or, for example, if the notes by the initial second opinion pathologist recommend seeking a further second opinion from a particular third registered user), and each new second opinion selection will appear as a new column. At the top of the column the clinicians' name will appear to designate the choices of that respective clinician.

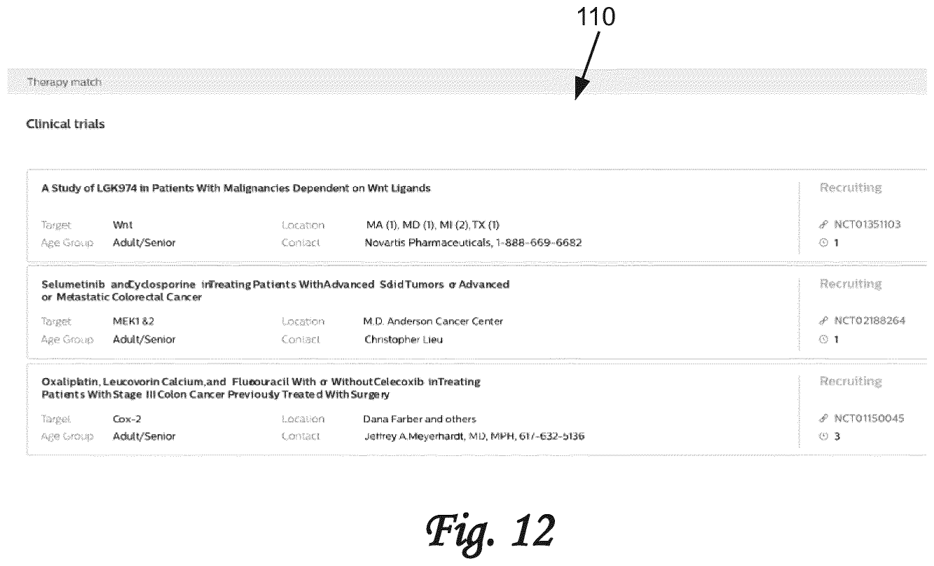

With reference back to FIG. 5 and with further reference to FIG. 12, an illustrative user interface display for displaying results produced by the at least one trial matching service provider 20₅ is shown. After filtering and variant prioritization, the clinical genomic data processing device automatically calls an API and invokes the clinical trial matching microservice 20₅. As already described with reference to FIG. 5, the clinical trial matching microservice 20₅ uses natural language processing to match variants to a database 70 of clinical trials. It associates clinical trials relevant for an individual patient based on the genomic aberrations within the tumor of that particular patient. FIG. 12 shows an illustrative example of a list 110 of such relevant clinical trials.

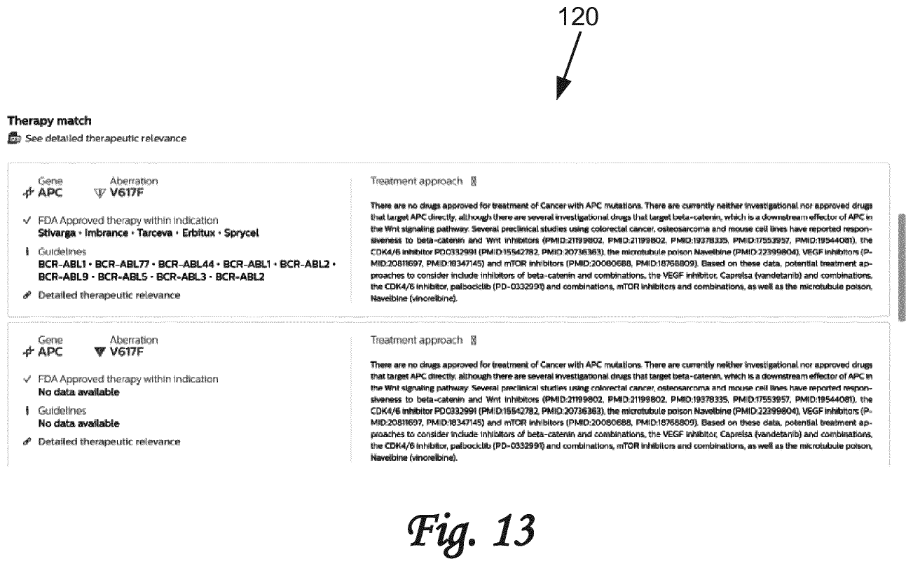

With reference now to FIG. 13, an illustrative user interface display for displaying results produced by at least one therapy matching service provider is shown. In parallel with (or instead of) the clinical trial matching, the system automatically calls an API and performs a microservice for therapy matching. This microservice operates analogously to the clinical trial matching microservice 20₅ but matches to a database of available clinical therapies for therapy matching, and may use a local or a remote database with manually curated genes and gene variants for which associations exists in the form of published clinical evidence. The evidence may come from clinical guidelines or published scientific or clinical journals. The association between a genomic aberration and therapy could be a positive one where the patient with a particular mutation may have increased response, or just the opposite. FIG. 13 shows a list 120 of such relevant therapy matches.

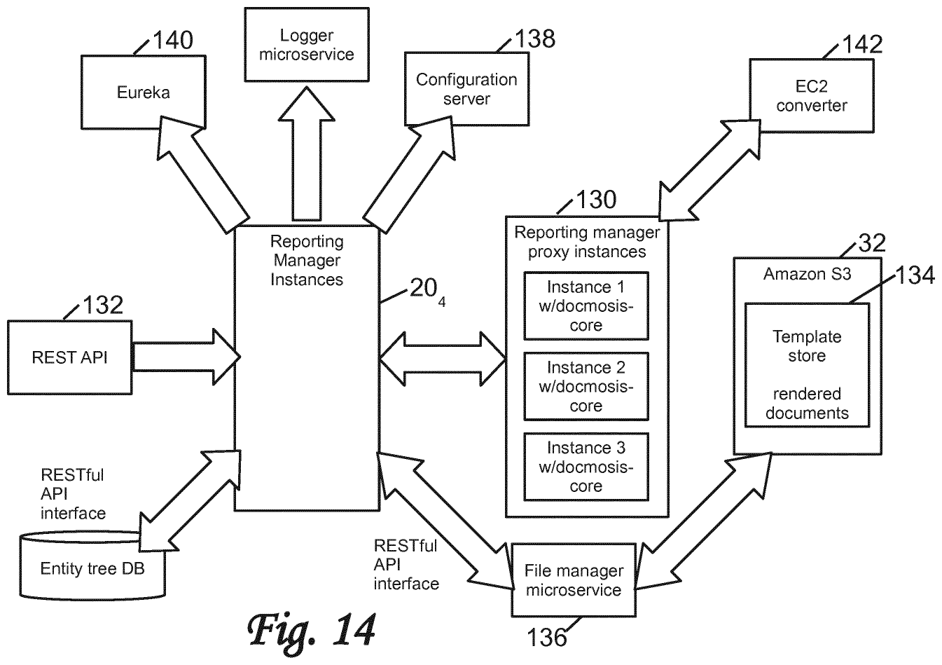

With reference to FIG. 14, an illustrative embodiment of the at least one reporting service provider 20₄ is described. The automated reporting microservice 20₄ for clinical reporting may be implemented by reporting manager instances 130. The report manager 20₄ is called by a REST API 132, and converts already-selected variants with their associated therapy matches (e.g. clinical phenotypes based on published existing clinical evidence) with clinical trial matches. The report manager 20₄ operates to collect the data for insertion into a document (i.e. clinical report). When populating templates e.g., stored in a template store 134 on the cloud-based storage 32 (e.g. Amazon Simple Storage Service, S3, in the illustrative embodiment) and accessed by a file manager microservice 136 as shown in FIG. 14, the structure of the data may be designed to match the structure of the template. At the start, the Reporting Manager process receives the following environment variables: (1) a Config server URI pointing to a configuration server 138; (2) a web server port number; and (3) a port number to provide to service discovery 140, which should be the same as the web server port. Next, the Reporting Manager 20₄ boots and accesses the config server 138 to get its configuration which will include: (1) the service discovery server 140 (e.g. Eureka) location (2) the service-name; (3) a Docmosis Key; (4) a Docmosis Converter Location (Static IP); (5) a service Description; (6) a cloud bucket; and (7) cloud bucket credentials. In the illustrative examples, various of these processes are performed using an Amazon Elastic Compute Cloud (Amazon EC2) converter 142. The reporting manager microservice 20₄ will then register itself with the Eureka server 138 as genomics-reporting-manager. This is an exemplary embodiment for creating the final report. Advantageously, the reporting process executes without the user having to cut-copy paste information and ensures fidelity of information.

The invention has been described with reference to the preferred embodiments. Modifications and alterations may occur to others upon reading and understanding the preceding detailed description. It is intended that the invention be construed as including all such modifications and alterations insofar as they come within the scope of the appended claims or the equivalents thereof.

1. A clinical genomic data processing device comprising:
at least one microprocessor; and
a non-transitory storage medium storing:
instructions readable and executable by the at least one microprocessor to implement a user interface configured to receive requests for execution of genomic workflows and to display output generated by the execution of the genomic workflows;
instructions readable and executable by the at least one microprocessor to implement a genomic workflow manager configured to manage an asynchronous messaging queue and to manage the execution of the genomic workflows; and
instructions readable and executable by the at least one microprocessor to implement service providers configured to perform jobs associated with the genomic workflows;
wherein the genomic workflow manager is configured to communicate with the service providers by messages exchanged via the asynchronous messaging queue to manage the execution of the genomic workflows via jobs performed by the service providers; and
wherein the service providers include:
at least one genomic processing service provider configured to perform a job comprising processing genomic data to generate a list of aberrations,
at least one annotation service provider configured to perform a job comprising processing a list of aberrations to generate annotated aberrations,
at least one aberration prioritization service provider configured to perform a job comprising processing a list of annotated aberrations to generate a prioritized list of annotated aberrations, and
at least one reporting service provider configured to perform a reporting job comprising at least display of a list of annotated aberrations via the user interface and receipt of a clinical report via the user interface.

2. (canceled)

3. The clinical genomic data processing device of claim 1 wherein:
the non-transitory storage medium further stores a list of registered users of the clinical genomic data processing device;
the reporting job comprises display of the list of annotated aberrations to a first registered user via the user interface and receipt of the clinical report from the first registered user via the user interface; and
the reporting job further comprises sending a request for a second opinion from the first registered user to a second registered user different from the first registered user via the user interface and display of the list of annotated aberrations to the second registered user via the user interface and receipt of a second opinion from the second registered user via the user interface and display of the second opinion to the first registered user via the user interface.

4. The clinical genomic data processing device of claim 1 wherein the service providers further include at least one of:
at least one trial matching service provider configured to perform a job comprising comparing the list of annotated aberrations to at least one clinical trial database to generate at least one clinical trial recommendation; and
at least one therapy matching service provider configured to perform a job comprising comparing the list of annotated aberrations to at least one clinical therapy database to generate at least one clinical therapy recommendation.

5. The clinical genomic data processing device of claim 1 wherein the genomic workflow manager is configured to maintain state machines representing the genomic workflows and is configured to update states of the state machines in accord with messages received from the service providers via the asynchronous messaging queue indicating successful completion of jobs performed by the service providers.

6. The clinical genomic data processing device of claim 5 wherein the service providers are stateless and receive all data needed for performing the jobs associated with the genomic workflows from the genomic workflow manager via the asynchronous messaging queue.

7. The clinical genomic data processing device of claim 1 wherein the user interface comprises web-based user interface including a webserver configured to execute computations to manage receipt of the requests for execution of genomic workflows and to generate visualizations of the output generated by the execution of the genomic workflows for display by a web client.

8. The clinical genomic data processing device of claim 1 wherein the service providers are microservices.

9. The clinical genomic data processing device of claim 1 further comprising:
a genetic sequencer;
wherein the service providers include at least one genomic processing service provider configured to perform a job comprising processing genomic data output by a sequencing run performed by the genetic sequencer to generate a list of aberrations.

10. The clinical genomic data processing device of claim 1 wherein the at least one microprocessor comprises a cloud based platform.

11. A non-transitory storage medium storing instructions readable and executable by at least one microprocessor to perform clinical genomic data processing, the instructions including:
instructions readable and executable by the at least one microprocessor to implement a user interface configured to receive requests for execution of genomic workflows and to display output generated by the execution of the genomic workflows;
instructions readable and executable by the at least one microprocessor to implement a genomic workflow manager configured to manage an asynchronous messaging queue and to manage the execution of the genomic workflows; and
instructions readable and executable by the at least one microprocessor to implement service providers configured to perform jobs associated with the genomic workflows, the service providers including at least one genomic processing service provider configured to perform a job comprising processing genomic data to generate a list of aberrations, at least one annotation service provider configured to perform a job comprising processing a list of aberrations to generate annotated aberrations, at least one aberration prioritization service provider configured to perform a job comprising processing a list of annotated aberrations to generate a prioritized list of annotated aberrations, and at least one reporting service provider configured to perform a reporting job comprising at least display of a list of annotated aberrations via the user interface and receipt of a clinical report via the user interface;
wherein the genomic workflow manager is configured to communicate with the service providers by messages exchanged via the asynchronous messaging queue to manage the execution of the genomic workflows via jobs performed by the service providers.

12. The non-transitory storage medium of claim 11 wherein the reporting job comprises display of the list of annotated aberrations to a first registered user via the user interface and receipt of the clinical report from the first registered user via the user interface, and the reporting job further comprises:
sending a request for a second opinion from the first registered user to a second registered user different from the first registered user via the user interface and display of the list of annotated aberrations to the second registered user via the user interface and receipt of a second opinion from the second registered user via the user interface and display of the second opinion to the first registered user via the user interface.

13. The non-transitory storage medium of claim 11 wherein the service providers further include at least one of:
at least one trial matching service provider configured to perform a job comprising comparing the list of annotated aberrations to at least one clinical trial database to generate at least one clinical trial recommendation; and
at least one therapy matching service provider configured to perform a job comprising comparing the list of annotated aberrations to at least one clinical therapy database to generate at least one clinical therapy recommendation.

14. The non-transitory storage medium of claim 11 wherein the genomic workflow manager is configured to maintain state machines representing the genomic workflows and is configured to update states of the state machines in accord with messages received from the service providers via the asynchronous messaging queue indicating successful completion of jobs performed by the service providers.

15. The non-transitory storage medium of claim 14 wherein the service providers are stateless and receive all data needed for performing the jobs associated with the genomic workflows from the genomic workflow manager via the asynchronous messaging queue.

16. The non-transitory storage medium of claim 11 wherein the user interface comprises web-based user interface including:
a webserver configured to execute computations to manage receipt of the requests for execution of genomic workflows and to generate visualizations of the output generated by the execution of the genomic workflows for display by a web client.

17. The clinical genomic data processing device of claim 11 wherein the service providers are microservices.

18. A clinical genomic data processing method comprising:
via a web-based user interface, receiving requests for execution of genomic workflows and displaying output generated by the execution of the genomic workflows;
via service providers implemented on a cloud-based platform comprising microprocessors, asynchronously performing jobs associated with the genomic workflows;
via a genomic workflow manager implemented on the cloud-based platform, maintaining state machines representing the genomic workflows and communicating with the service providers by messages exchanged via an asynchronous messaging queue to manage the execution of the genomic workflows via the jobs asynchronously performed by the service providers and updating states of the state machines in accord with messages received from the service providers via the asynchronous messaging queue indicating successful completion of the jobs performed by the service providers;
wherein the asynchronous performing of jobs associated with the genomic workflows include:
via at least one genomic processing service provider, performing a job comprising processing genomic data to generate a list of aberrations;
via at least one annotation service provider, performing a job comprising processing a list of aberrations to generate annotated aberrations;
via at least one aberration prioritization service provider, performing a job comprising processing a list of annotated aberration's to generate a prioritized list of annotated aberrations; and
via at least one reporting service provider, performing a reporting job comprising at least display of a list of annotated aberrations via the web-based user interface and receipt of a clinical report via the web-based user interface.

19. (canceled)

20. The clinical genomic data processing method of claim 18 wherein the reporting job comprises display of the list of annotated aberrations to a first registered user via the web-based user interface and receipt of the clinical report from the first registered user via the web-based user interface, and the reporting job further comprises:
sending a request for a second opinion from the first registered user to a second registered user different from the first registered user via the web-based user interface, and display of the list of annotated aberrations to the second registered user via the web-based user interface and receipt of a second opinion from the second registered user via the web-based user interface and display of the second opinion to the first registered user via the web-based user interface.

21. The clinical genomic data processing method of claim 18 wherein the asynchronous performing of jobs associated with the genomic workflows further include at least one of:
via at least one trial matching service provider, performing a job comprising comparing the list of annotated aberrations to at least one clinical trial database to generate at least one clinical trial recommendation; and
via at least one therapy matching service provider, performing a job comprising comparing the list of annotated aberrations to at least one clinical therapy database to generate at least one clinical therapy recommendation.

22. The clinical genomic data processing method of claim 18 wherein the service providers are stateless and receive all data needed for performing the jobs associated with the genomic workflows from the genomic workflow manager via the asynchronous messaging queue.
